(19) United States
(12) Patent Application Publication
(10) Pub. No.: US 2023/0173239 A1
(43) Pub. Date: Jun. 8, 2023

(54) LUMEN-APPOSING SHUNT DEVICE TRANSPORTING FLUID BETWEEN TWO BODY CAVITIES

(71) Applicant: Kyongtae Bae, Henderson, NV (US)

(72) Inventors: Kyongtae Bae, Henderson, NV (US); Junu Bae, Henderson, NV (US); Sonu Bae, Henderson, NV (US); Choonkyu Kim, Gyeonggi-do (KR)

(21) Appl. No.: 17/542,839

(22) Filed: Dec. 6, 2021

(51) Int. Cl. A61M 27/00; A61M 25/09

(52) U.S. Cl. CPC A61M 27/008; A61M 25/09; A61M 27/006; A61M 2025/09008; A61M 2025/09166; A61M 2027/004; A61M 2210/1085

(57) ABSTRACT

Lumen-apposing shunt devices for transporting body fluid from a first compartment (e.g., peritoneal cavity) to a second compartment (e.g., bladder) comprise a longitudinal tube communicating between the two compartments and retention systems that separate apart and maintain each end of the tube within the respective compartment. The retention systems include double self-expandable wheels and double inflatable balloons enveloping the tube transversely, with one wheel or balloon in the first compartment and the other wheel or balloon in the second compartment. The self-expanded wheels or inflated balloons support and retain the shunt device between the compartments as fluid (e.g., ascites) from the first compartment flows into the second compartment in response to pressure gradients between the compartments. The shunt device contains a one-way valve mechanism to provide unidirectional flow of fluid from the first to the second compartment and to prevent reflux from the second to the first compartment.

### TECHNICAL FIELD

This invention relates to lumen-apposing shunt systems for transporting fluid between two body cavities. More specifically, the present invention pertains to lumen-apposing peritoneo-vesicular shunt systems for unidirectional flow of ascitic fluid into the bladder.

### BACKGROUND ART

A number of medical conditions could lead to chronic excess fluid collections in different body compartments, including urinary bladder distension secondary to lower urinary tract obstruction, pleural effusion, pericardiac effusion, hydrocephalus, and ascites. Among these fluid collections, ascites accumulates in the peritoneal cavity in patients with a range of pathologies, including liver disease, cancer, congestive heart failure, and kidney disease. Ascites is the leading cause of hospitalization and poor quality of life in these patients. As the volume of ascites increases, patients suffer from abdominal pain, bloating, expansion of abdominal girth and size, and shortness of breath. Appropriate clinical management of ascites is crucial to minimize complications and improve quality of life. Ascites that cannot be managed by medical therapy is called refractory ascites and is associated with poor survival and persistently worsens the quality of life (1).

The initial approach for treating ascites is dietary salt restriction and use of diuretics. However, up to 10% of patients develop tolerance and become refractory to diuretic therapy despite maximum use of diuretics. When pharmaceutical therapy alone is insufficient, interventional therapy is performed; this can be classified into two categories: (1) periodic external drainage of ascites, paracentesis, or (2) constant internal drainage or decompression of ascites to another body cavity. Paracentesis involves identifying a pocket of ascites using ultrasound imaging, inserting a largebore needle and plastic sheath through the abdominal wall into the peritoneal cavity, and removing the needle while leaving the sheath through which ascites drains. Paracentesis is an outpatient procedure that does not require sedation, and patients are promptly discharged following the procedure. Although paracentesis can be easily performed and is usually safe, it is a temporary repair and must be repeated periodically every 2-3 weeks. Repeated paracentesis is costly and may expose patients to serious complications including bleeding and infection. Paracentesis greater than 5 L of ascites is often followed by the intravenous infusion of albumin to reduce the risk of post-paracentesis circulatory dysfunction, a serious clinical condition leading to increased risk of hospitalization and mortality. Furthermore, because paracentesis is a temporary relief, patients have to endure symptoms as ascites constantly reaccumulates and expands between periodical paracenteses.

One surgical or interventional radiological approach to eliminating ascites is a peritoneo-venous shunt system (Denver shunt), which drains ascites into the superior vena cava via a subcutaneously implanted unidirectional pump (2-4). One end of the pump is connected to a multi-perforated intra-peritoneal tube, while the other end is a subcutaneous tube reaching the superior vena cava through the internal jugular vein. The valve must be manually pumped daily by the patient or caregiver to prevent fibrous particles from adhering to the catheter and causing obstruction (5). In current clinical practice, the peritoneo-venous shunt has been largely discontinued, because of serious complications including infection, coagulopathy, and venous occlusion (6). Another approach performed by interventional radiologists is transjugular intrahepatic portosystemic shunt (TIPS), which involves the creation of a shunt between portal and suprahepatic veins and the deployment of an expandable metal stent within the shunt. TIPS essentially bypasses the cirrhotic hepatic resistance and decompresses the portal hypertension, thereby reducing the recurrence of ascites (7). However, the benefits of TIPS are somewhat offset by the increased incidence of new or worsening hepatic encephalopathy following TIPS. TIPS is also not recommended in patients with severe liver disease, progressive renal failure, hepatic encephalopathy, or severe cardiopulmonary disease (1).

An alternative approach to eliminating ascites is to drain ascitic fluid into the bladder rather than into the venous blood. The first published peritoneal-urinary drainage system consisted of a squeeze-bulb and two tubes (8). The squeeze-bulb serving as a pump was implanted subcutaneously and connected to the peritoneal cavity and urinary bladder via the two tubes. External hand compression of the skin over the bulb propelled ascites into the urinary bladder. Each tube had a one-way valve to maintain unidirectional flow of the ascitic fluid from the peritoneal space to the bladder. Ascites drained to the bladder was eliminated through urination. Similar to this manual squeeze-bulb peritoneal-urinary drainage system, a more advanced system with a rechargeable battery-powered pump was developed and has become commercially available since 2011 (Alfapump system, Sequana Medical AG, Zurich, Switzerland) (U.S. Pat. No. US9149613B2) (9). The Alfapump system consists of a subcutaneously implanted battery-powered pump connected to the peritoneal cavity and urinary bladder via two tubes. The pump allows ascites to continuously drain from the peritoneal cavity into the urinary bladder. The system has pressure-monitoring sensors and is programmable to specify volume and time of infusion. The pump and tubes are surgically placed under general anesthesia. The Alfapump system was evaluated in recent clinical trials and found to be effective for reducing the need for paracentesis and improving quality of life in patients with refractive ascites (10, 11). However, there remained a number of adverse events related to the surgical procedure and the Alfapump system, requiring careful patient selection and postoperative monitoring to balance the benefit against the invasiveness and frequent complications of the system (1).

A variation of the peritoneal-urinary drainage system is to directly shunt ascites from the peritoneum to the bladder without a pump that connects and bypasses the two body compartments. A few devices were described in recently published patents (US8394048, US20140012180A1, WO 2018/071373 Al) (12, 13). The proposed devices included a one-way valve mechanism to enforce unidirectional flow of fluid and prevent reflux of urine. The devices were secured in the bladder wall by different types of anchorage mechanisms, such as a flange or plug that seats on and braces both edges of the bladder walls containing the shunt opening. Compared to these bracket-like anchorage mechanisms that directly lock onto the bladder wall, in our approach we use a lumen-apposing retention means that does not directly brace the bladder wall but rather peripherally apposes to the surface of the bladder wall. Our lumen-apposing retention system is based on the consideration that the bladder wall is not a fixed rigid structure, but a muscle that constantly and dynamically varies in shape and size as urine accumulates and dissipates from the bladder.

An additional important consideration in designing a shunt device is how to access body cavities and implant the shunt device across the body cavities. A device is designed to be implanted either surgically by a surgeon through an opening made in the abdominal cavity or transurethrally without surgery by means of a highly sophisticated transurethral cystoscopic system that may be able to introduce the bladder wall anchorage device. However, the non-surgical transurethral implantation of the shunt devices, particularly regarding how to implant bulky bladder anchorage components, remains a major practical and technical challenge.

As patients with ascites are often in debilitated health conditions and have limited tolerance in extensive surgical procedures, it is highly desirable to implement a safe, non-invasive, and efficient treatment method without requiring a prolonged risky surgical operation. Therefore, it is essential to develop a device that is highly effective in the treatment of ascites and is relatively easily implantable using a minimally invasive interventional radiological or surgical procedure. Additional practical features of a device would include simplicity in operation (drainage of ascites on the basis of gravity and/or contraction of patient’s voluntary muscles without requiring complex programming or pumping system) and the easy removal of the device when clinically indicated.

### DISCLOSURE

### Technical Solution

In the first aspect of the present invention, a shunt device transporting fluid between two body cavities is provided which comprises:
a lumen-apposing retention system comprising double retention means which is expandable wherein one of the retention means expands to appose to a lumen of a first body cavity and the other retention means expands to appose to a lumen of a second body cavity;
a hollow drainage tube, extending between the first body cavity and the second body cavity, wherein each end of the drainage tube is retained by each of retention means; and
a drainage valve, communicating with the lumen of the drainage tube, wherein the drainage valve is a one-way valve permitting fluid flow from the first body cavity to the second body cavity and preventing fluid flow from the second body cavity to the first body cavity;
wherein a bulk size of the expanded retention means at either side of the body cavities is larger than the cross-sectional coverage of the hollow drainage tube over a shunt aperture area across the two body cavities such that the expanded retention means prevents the hollow drainage tube from slipping out of the shunt aperture into either side of the body cavities.

The retention means may contain fiducial markers including radio-opaque fiducial markers for tracking the location and position of the retention means.

In one embodiment, the retention means may be double self-expandable wheels.

Here, this type of the shunt device is called the self-expandable double-wheel shunt device.

In another embodiment, the retention means may be double inflatable balloons, and the shunt device further comprises:
a small-caliber balloon lumen extending and communicating with the balloons that are inflatable by infusing air or fluid using a small-caliber balloon tube; and
a balloon valve, communicating with the small-caliber balloon lumen, wherein the balloon valve is a one-way valve allowing air or fluid flow to inflate the balloons and preventing a deflation of the balloons once inflated.

Here, this type of the shunt device is called the inflatable double-balloon shunt device.

Wherein, the self-expandable wheels may be composed of biocompatible polymer or silicon.

Wherein, the hollow drainage tube may comprise a peritoneal portion located in the peritoneal cavity and a bladder portion located in the bladder.

Wherein, the peritoneal portion of the hollow drainage tube may comprise one or more holes to enhance the drainage of ascites.

Depending on clinical needs, a shunt device is applicable to transporting various type of body fluid between two body cavities. For ascites, the first body cavity is the peritoneal space while the second body cavity is the bladder. For fetal urinary fluid secondary to lower urinary tract obstruction, the first body cavity is the fetal bladder while the second body cavity is the maternal amniotic sack. For pleural effusion, the first body cavity is the pleural space while the second body cavity is the peritoneal space. For pericardiac effusion, the first body cavity is the pericardiac space while the second body cavity is the pleural space. For a pancreatic cystic fluid collection, the first body cavity is a pancreatic cyst while the second body cavity is the stomach. For a visceral fluid collection, the first body cavity is a visceral fluid collection pocket while the second body cavity is the gastric or intestinal or bladder lumen. For the purpose of draining a body fluid collection to outside of the body, the first body cavity is a body fluid collection pocket while the second body cavity is an external drainage bag outside of the body. In some clinical needs including a continued washout of a body cavity or enteric nutritional support rather than drainage, the aforementioned first body and second cavities may be switched with reversed fluid flow direction.

The shunt device can be implanted in a body of patient, via a transurethral, transvesicular, or transabdominal approach, depending on the indication of clinical applications and accessibility of the device.

In the second aspect of the present invention, a combined assembly for implanting a shunt device is provided.

In one embodiment, the shunt device of the combined assembly is the self-expandable double-wheel shunt device, and the combined assembly for implanting the self-expandable double-wheel shunt device comprises:
a guide catheter which is steerable to place a tip of the catheter to a target wall(s) of the body cavities that is(are) a target for the implantation of a shunt device;
a trocar which is inserted and advanced through a lumen of the guide catheter to puncture the target wall(s) of the body cavities;
the shunt device which is introduced through the lumen of the guide catheter to the target wall(s) of the body cavities; and
a pusher catheter which pushes the shunt device forward to slide through the lumen of the guide catheter.

In one embodiment, the shunt device of the combined assembly is the inflatable double-balloon shunt device, and the combined assembly for implanting the inflatable double-balloon shunt device comprises:
a guide catheter which is steerable to place a tip of the catheter to a target wall(s) of the body cavities that is(are) a target for the implantation of a shunt device;
a trocar which is inserted and advanced through a lumen of the guide catheter to puncture the target wall(s) of the body cavities;
the shunt device which is introduced through the lumen of the guide catheter to the target wall(s) of the body cavities;
a pusher catheter which pushes the shunt device forward to slide through the lumen of the guide catheter; and
a balloon inflation feeding tube which extends from outside of a patient to an entrance of the small-caliber balloon lumen, wherein the balloon inflation feeding tube fits into the small-caliber balloon lumen to inflate balloons of the shunt device by infusing fluid or air using a syringe.

The combined assembly may further comprise a guide wire which is introduced through or laterally along the hollow lumen of the trocar to serve as a cannulation track for a shunt device to move across the body cavities.

Wherein, the guide catheter may contain fiducial markers including radio-opaque fiducial markers to track the location and position of the guide catheter.

In the third aspect of the present invention, a method to implant a peritoneo-vesicular shunt device from the combined assembly via urethra is provided.

In one embodiment, the shunt device of the combined assembly is the self-expandable double-wheel shunt device, and the method to implant the self-expandable double-wheel peritoneo-vesicular shunt device from the combined assembly comprises:
placing the guide catheter in a bladder and steering the tip of the guide catheter to a bladder wall that is a target for the implantation of the shunt device and that juxtaposes a peritoneum in direct contact with ascites within a peritoneal space;
introducing and advancing the trocar through the guide catheter to the target bladder wall and puncturing the target bladder wall;
introducing and pushing the shunt device forward by the pusher catheter through the lumen of the guide catheter to advance and expose only a peritoneal portion of the shunt device out of the guide catheter into a peritoneal cavity, which allows the folded peritoneal wheel of the shunt device to unbind and self-expand;
pulling back slightly the assembly of the guide catheter, pusher catheter, and shunt device to place the tip of the guide catheter within the bladder while maintaining the expanded peritoneal wheel to appose to the bladder wall;
withdrawing the guide catheter slightly while keeping the pusher catheter stationary to uncover and unbind a bladder portion of the shunt device out of the guide catheter in the bladder, which allows the folded bladder wheel of the shunt device to unbind and self-expand so that the shunt device is stabilized and secured across the bladder wall; and
removing the pusher catheter, trocar, and guide catheter.

In another embodiment, the shunt device of the combined assembly is the inflatable double-balloon peritoneo-vesicular shunt device, and the method to implant the inflatable double-balloon peritoneo-vesicular shunt device from the combined assembly comprises:
placing the guide catheter in a bladder and steering the tip of the guide catheter to a bladder wall that is a target for the implantation of the shunt device and that juxtaposes a peritoneum in direct contact with ascites within a peritoneal space;
introducing and advancing the trocar through the guide catheter to the target bladder wall and puncturing the target bladder wall;
introducing and pushing the shunt device forward by the pusher catheter through the lumen of the guide catheter to advance and expose only a peritoneal portion of the shunt device out of the guide catheter into a peritoneal cavity, which allows a peritoneal balloon of the double-balloon shunt to unbind;
withdrawing the guide catheter slightly while keeping the pusher catheter stationary to uncover and unbind a bladder balloon of the shunt device out of the guide catheter in the bladder;
inflating the bladder balloon and peritoneal balloon of the shunt device by infusing fluid or air via the balloon inflation feeding tube to stabilize and secure the shunt device across the bladder wall;
removing the balloon inflation feeding tube from the balloon valve that closes the small-caliber balloon lumen; and
removing the pusher catheter, trocar, and guide catheter.

The method to implant a peritoneo-vesicular shunt device from the combined assembly via urethra may further comprise:
introducing and advancing a guide wire through or laterally along the hollow lumen of the trocar to serve as a cannulation track for the shunt device to access the peritoneal space, after the puncturing the target bladder wall, and
removing the guide wire, after the shunt device being secured across the bladder wall.

In the fourth aspect of the present invention, a method to remove the implanted shunt device is provided.

In one embodiment, the shunt device of the combined assembly is the self-expandable double-wheel shunt device, and the method to remove the implanted double-wheel shunt device comprises:
accessing and grasping a side of the shunt device located in the second body cavity by using a grasping forceps;
pulling the hollow drainage tube of the shunt device to slip through the shunt implant site into the second body cavity and forcing the expanded wheel of the shunt device in the first body cavity to flip and reverse its orientation and slip through the shunt implant site into the second body cavity; and
further pulling and removing the freed shunt device from the second body cavity.

In another embodiment, the shunt device of the combined assembly is the inflatable double-balloon shunt device, and the method to remove the implanted double-balloon shunt device comprises:
accessing and snipping the balloon of the shunt device located in the second body cavity by using a biopsy or snipping tool to deflate the balloons in both body cavities;
grasping and pulling the deflated and freed shunt device by using a grasping forceps to slip through the shunt implant site from the first body cavity to the second body cavity; and
further pulling and removing the released shunt device from the second body cavity.

### DESCRIPTION OF DRAWINGS

FIG. 1 is an illustration of a male patient with the implantation of a self-expandable double-wheel shunt device and an inflatable double-balloon shunt device of the present invention.

FIG. 2 is a schematic view of a self-expandable double-wheel shunt device in a folded configuration and in an expanded configuration.

FIGS. 3 and 4 are schematic views of self-expandable double-wheel shunt devices of two different designs in a folded configuration and in an expanded configuration.

FIG. 5A is a schematic view of an inflatable double-balloon shunt device in a deflated configuration and in an inflated configuration.

FIG. 5B, FIG. 5C and FIG. 5D are transverse cross-sections of the device of FIG. 5 taken along lines A-A, B-B, and C-C, respectively.

FIG. 6 is an illustration of a male patient with a guide catheter inserted in the bladder cavity through the urethra to provide transurethral access for the implantation of the shunt device.

FIG. 7 is a cross-sectional view of a combination of a folded shunt device, trocar, and pusher catheter inserted through the guide catheter, where a target portion of the bladder wall is punctured by the trocar.

FIG. 8 is a cross-sectional view of the guide catheter, the trocar, the pusher catheter, and a double-wheel shunt device with its bladder-wheel folded but peritoneal-wheel expanded, implanted through the bladder wall into the peritoneal space.

FIG. 9 is an illustration of a slight withdrawal of the assembly of the guide catheter, trocar, push catheter, and shunt device shown in FIG. 8 to pull the expanded peritoneal wheel to appose to the bladder wall.

FIG. 10 is an illustration of pulling back of the guide catheter while keeping the push catheter stationary to uncover and unbind the self-expandable bladder wheel of the double-wheel shunt device.

FIG. 11 shows a sequence of implantation in that the wheels of a double-wheel shunt device expand step-by-step to appose to a single-layer wall.

FIG. 12 shows a sequence of implantation in that the wheels of a double-wheel shunt device expand step-by-step to appose to a double-layer wall.

FIG. 13 is a cross-sectional view of a guide catheter, a trocar, a pusher catheter, a balloon inflation tube, and a double-balloon shunt device implanted through the bladder wall, following a partial withdrawal of the guide catheter to expose both balloons of the double-balloon shunt device in a deflated configuration.

FIG. 14 is an illustration of a double-balloon shunt device with its balloons inflated to stabilize and retain the shunt device across the bladder wall, following the inflation of the balloons and the removal of the balloon inflation tube, pusher catheter, trocar, and guide catheter.

FIG. 15 is an illustration of a double-wheel shunt device being pulled toward the second body cavity (e.g., bladder), forcing its expanded wheel in the first body cavity (e.g., peritoneal space) to flip and reverse its orientation, so that it can slip through the shunt implant site together with the hollow drainage tube into the second body cavity.

### Best Mode

Some embodiments of the present invention are peritoneo-vesicular shunt devices that are designed to be implanted through the urethra and retained across the bladder wall to permit the drainage of ascites into the bladder.

The inferior aspect of the peritoneum abuts the urinary bladder. Ascites accumulates in the peritoneal cavity. When a shunt is implanted between the peritoneal cavity and bladder, ascites drains via the shunt into the bladder and is subsequently eliminated by urination. The flow of ascites is facilitated by the force of gravity or increased intra-abdominal pressure due to increased volume of ascites or patient’s tensing of abdominal muscles. No external mechanical pump is necessary to move ascites from the peritoneal cavity to bladder.

The shunt device is placed at a location where the peritoneum and bladder wall anatomically juxtapose to promptly facilitate the creation of a peritoneo-vesicular shunt.

The shunt device is configured in two types: a self-expandable double-wheel type and an inflatable double-balloon type.

The self-expandable double-wheel shunt device consists of a hollow, cylindrical column with double wheels to provide secure retention at the peritoneum and bladder wall interface. After the placement of the self-expandable double-wheel shunt device, the double wheels that envelope transversely both ends of the shunt device are self-expanded, with one wheel within the peritoneal cavity and the other within the bladder lumen, thereby retaining the device across the bladder wall as a peritoneo-vesicular shunt.

The inflatable double-balloon shunt device consists of a hollow, cylindrical column with double balloons to provide secure retention at the peritoneum and bladder wall interface. After the placement of the device, double balloons enveloping the transverse mid-body of the device are inflated, with one balloon within the peritoneal cavity and the other within the bladder lumen, thereby retaining the device across the bladder wall as a peritoneo-vesicular shunt device.

In both types of the shunt device, the proximal uptake portion of the device extends into the peritoneal cavity to be in communication with ascites, while the distal portion of the device extends into the bladder. The shunt device contains a one-way valve mechanism (e.g., duckbill valves) to provide unidirectional flow of the fluid from the peritoneal cavity to the bladder and to prevent reflux of urine into the peritoneal cavity.

The peritoneo-vesicular shunt devices described in detail in the present embodiment are for implantation through a transurethral route without requiring cutting skin or surgery. However, the devices may be implanted using a transvesicular or transabdominal route with or without guidance of various imaging systems including ultrasound, CT, MRI, fluoroscopy, cystoscopy, and laparoscopy.

The transurethral implantation of the device is performed by a series of catheter-based operations. The first step is an insertion of a semi-rigid guide catheter through the urethra into the bladder. The intravesicular tip of the guide catheter is placed and adjusted to abut the bladder wall at a target of the shunt device implantation that can be selected with guidance of imaging. The second step is an introduction of a trocar through the lumen of the guide catheter to puncture the target bladder wall to create a shunt pathway. The third step is pushing and advancing the shunt device using a pusher catheter through the guide catheter such that the peritoneal portion of the shunt device is uncovered and released out of the guide catheter into the peritoneal space.

In the self-expandable double-wheel shunt device, the freed peritoneal wheel expands by itself to be retained in the peritoneal space. After the shunt device is placed appropriately across the peritoneum and bladder wall interface, the fourth step is a slight withdrawal of the assembly of the guide catheter and shunt device to place the tip of the guide catheter within the bladder while maintaining the expanded peritoneal wheel to appose to the bladder wall. The fifth step is a partial pullback of the guide catheter while keeping the pusher catheter stationary to expose the bladder portion of the shunt device within the bladder.

In the double-wheel shunt device, the freed bladder wheel expands by itself to be retained in the bladder.

For the inflatable double-balloon shunt device, an additional step is required to inflate the double balloons of the shunt device to secure the device across the bladder wall. The inflation of the balloons is achieved by delivery of fluid or air via a long, small-caliber balloon inflation feeding tube which extends from outside of the patient to the entrance of the balloon inflation lumen of the shunt device. The balloon inflation feeding tube runs parallel to the course of the pusher catheter within the guide catheter. The entrance of the balloon inflation lumen of the shunt device is obstructed by a one-way balloon valve. The balloon inflation feeding tube is initially connected and inserted through the one-way balloon valve into the balloon inflation lumen. After the inflation of the balloons is achieved by infusion of air or fluid via the balloon inflation feeding tube, the balloon inflation tube is pulled away and removed. Once the shunt device is securely retained by either the expanded double-wheels or the inflated double-balloons, the guide catheter and pusher catheter are removed from the shunt device to complete the implantation process.

When it is desired to remove an implanted double-wheel shunt device, the shunt device in the bladder side is directly pulled and removed by using a grasping forceps via cystoscopy. For an implanted double-balloon shunt device, a biopsy or snipping tool via cystoscopy is introduced to snip and deflate the bladder balloon that sustains the shunt device at the bladder wall. With the deflation of both balloons, the shunt device is freed and removed by using a grasping forceps of a cystoscope.

### MODE FOR INVENTION

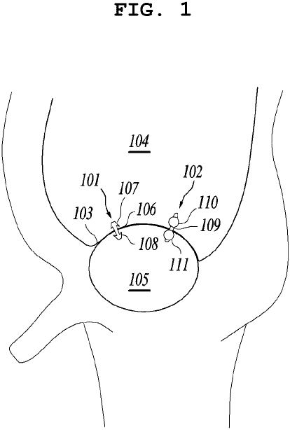

FIG. 1 shows one embodiment of a self-expandable double-wheel shunt device 101 and an inflatable double-balloon shunt device 102 implanted in the bladder wall 103. Ascites collected in the peritoneal cavity 104 is drained to the bladder 105 through the shunt devices. The self-expandable double-wheel shunt device 101 in the long-axis includes a hollow tubular structure 106 that has openings in the peritoneal cavity 104 and bladder 105 to provide a fluid passage for ascites to drain from the peritoneal cavity to the bladder. The tubular structure 106 includes a one-way drainage valve that is activated and opened by a pressure differential between the peritoneal cavity and bladder. The pressure differential is generated by a combination of gravity and contraction of muscles in the abdominal wall. The one-way drainage valve remains closed, preventing reflux of urine into the peritoneal cavity.

The self-expandable double-wheel shunt device 101 includes a pair of expanded wheels spaced apart such that the peritoneal wheel 107 is deployed within the peritoneal cavity 104 while the bladder wheel 108 is located within the bladder 105. The expanded wheels allow the shunt device to be retained across the bladder wall.

The inflatable double-balloon shunt device 102 contains a hollow tubular structure 109 whose structure and function are the same as the aforementioned tubular structure 106 to form a passage of ascites from the peritoneal cavity to the bladder. The inflatable double-balloon shunt device 102 includes a pair of inflatable balloons spaced apart such that the peritoneal balloon 110 is deployed within the peritoneal cavity 104 while the bladder balloon 111 deployed within the bladder 105. The inflated balloons secure the shunt device to be retained across the bladder wall.

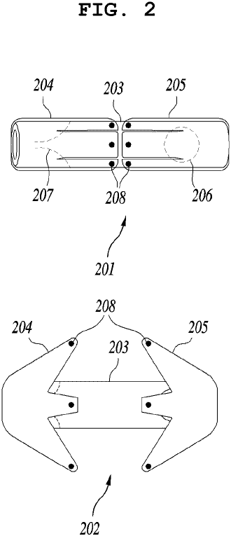

FIG. 2 shows one embodiment of a self-expandable double-wheel shunt device in a folded configuration 201 and in an expanded configuration 202. The shunt device in the long-axis consists of a hollow tube 203 with open ends for the drainage of ascites. The shunt device contains double wheels, 204 and 205, enveloping the outer walls of the hollow tube 203 at both ends. The double wheels, 204 and 205, are composed of elastic but relatively stiff biocompatible polymer or silicon such that the wheels are in a folded configuration when introduced through the lumen of a guide catheter but expand readily by themselves in a full-fledged configuration when uncovered and freed out of the lumen of the guide catheter. The wheel 204 is placed in the bladder, while the wheel 205 is located in the peritoneal space. The mid-portion of the hollow tube 203 is secured across the bladder wall. The longitudinal length of the hollow tube 203 between the expanded wheels 204 and 205 is in the range from 4 mm, which is the mean bladder wall thickness in adults, to 10 mm. While this dimension serves as a reference, the length and caliber of the tube and wheel are scaled and adjusted for the patient’s size and anatomy of interest in various clinical applications. The mid-portion tube is composed of stiff biocompatible polymer or silicon, or contains a metal spring to preserve an adequate passage of ascites against contractile force by the bladder wall muscle.

The hollow tube 203 contains side holes 206 to enhance the drainage of ascites. One-way valve (e.g., duckbill valve) 207 is located in the lumen of the tube 203 to allow unidirectional flow of ascites from the peritoneal cavity to the bladder and to prevent reflux of urine into the peritoneal cavity. One-way valve 207 is designed and configured to appropriately open and close in response to pressure gradients between the peritoneal cavity and bladder. Increased peritoneal pressure due to accumulated ascites or contraction of abdominal muscles will provide force to open the valves to drain ascites into the bladder. Valves remain closed without a positive pressure gradient exerted from the peritoneum to the bladder.

During or after the implantation of a shunt device, it is informative to track and monitor the location and configuration state of the shunt device in the body cavities. For this purpose, the hollow tube 203 is partially or entirely coated with barium sulfate. In addition, fiducial markers 208 are embedded in the shunt device. Fiducial markers made of radio-opaque materials including barium sulfate or metal can be detected under fluoroscopy or CT. Monitoring changes in the position of the hollow tube and fiducial markers in the wheels facilitate the evaluation of folded or expanded configuration of the shunt device.

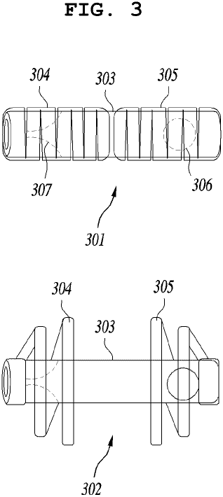

FIG. 3 shows another embodiment of a self-expandable double-wheel shunt device in a folded configuration 301 and in an expanded configuration 302. The design and configuration of the wheels are distinct from those of FIG. 2. However, the hollow tube 303, the side holes 306, and one-way valve 307 are the same design and function as the corresponding part described in FIG. 2.

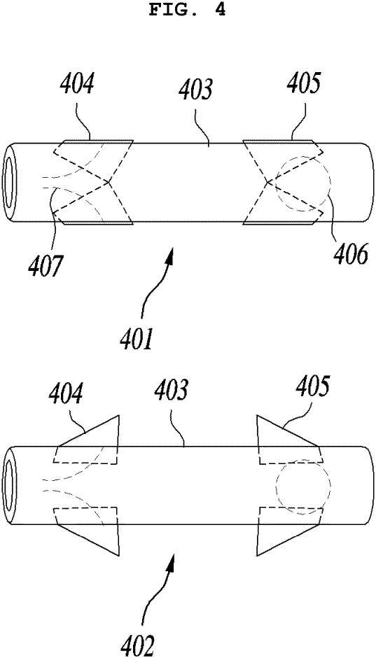

FIG. 4 shows another embodiment of a self-expandable double-wheel shunt device in a folded configuration 401 and in an expanded configuration 402. The design and configuration of the wheels are distinct from those of FIG. 2. However, the hollow tube 403, the side holes 406, and one-way valve 407 are the same design and function as the corresponding parts described in FIG. 2.

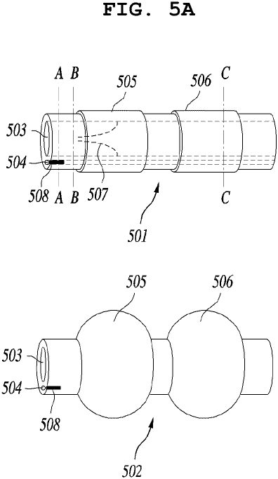

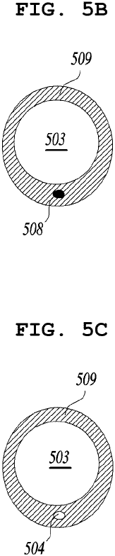

FIG. 5A shows an inflatable double-balloon shunt device in a deflated configuration 501 and in an inflated configuration 502. The double-balloon shunt device comprises two hollow lumens, a central lumen 503 for the drainage of ascites and a small-caliber balloon lumen 504 for the inflation of the balloons, 505 and 506, that envelop the outer walls of the shunt device.

One-way drainage valve (e.g., duckbill valve) 507 is located in the central lumen 503 to allow unidirectional flow of ascites from the peritoneal cavity to the bladder and to prevent reflux of urine into the peritoneal cavity. One-way valve 507 is designed and configured to appropriately open and close in response to pressure gradients between the peritoneal cavity and bladder. Increased peritoneal pressure due to accumulated ascites or contraction of abdominal muscles will provide force to open the valves to drain ascites into the bladder. Valves remain closed without a positive pressure gradient exerted from the peritoneum to the bladder.

Inflation and deflation of the balloons 505 and 506 is accomplished through the balloon lumen 504. The entrance of the balloon lumen 504 contains a one-way balloon valve (e.g., duckbill valve or elastic plug valve) 508 which keeps the balloon lumen 504 closed unless forcefully pushed apart to open.

The peritoneal balloon 506 envelops the peritoneal portion of the shunt device within the peritoneal cavity and secures the upstream drainage position of the shunt device. The bladder balloon 505 envelopes the bladder portion of the shunt device spaced apart from the peritoneal balloon 506 to ensure that the shunt device between the two balloons is lodged at the bladder wall. Both balloons are in communication with the common balloon lumen 504 for inflation and deflation of the balloons.

FIGS. 5B, 5C, and 5D show the transverse cross-section views of the shunt device 501 including the central lumen 503 for the drainage of ascites, the small-caliber balloon lumen 504 for the inflation of the balloons, and the one-way balloon valve 508. The central luminal wall 509 is composed of relatively stiff biocompatible polymer or silicon, or supported by a metal spring, while the balloon wall 510 is composed of readily inflatable biocompatible polymer or silicon.

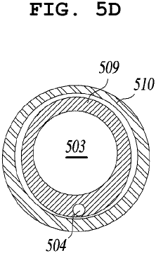

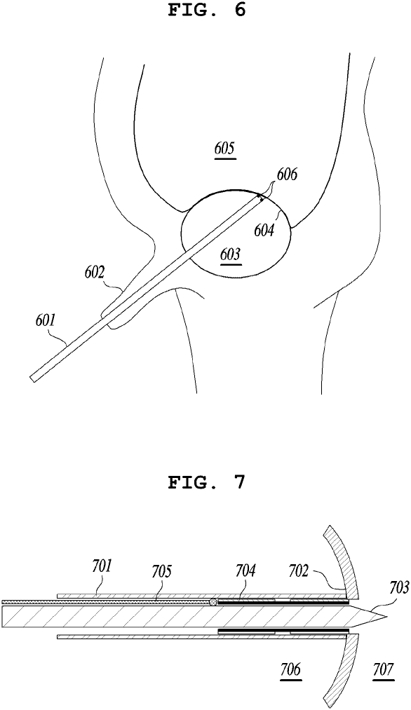

FIG. 6 illustrates a transurethral approach in a male patient having a guide catheter 601 inserted through a male urethra 602 in order to provide transurethral access for the implantation of the shunt device in the bladder 603. After the guide catheter 601 is introduced within the bladder 603, it is angled toward a suitable location of bladder wall 604 that juxtaposes the peritoneum directly in contact with ascites within the peritoneal space 605. The placement and steering of the guide catheter 601 can be directed using various imaging systems including fluoroscopy, ultrasound imaging, x-ray computed tomography, and magnetic resonance imaging. In the process of maneuvering the guide catheter, it is informative to track and monitor the location of the tip of the guide catheter in the body cavities. For this purpose, the guide catheter 601 is partially or entirely coated with barium sulfate, and fiducial markers 606 are embedded in the tip of the guide catheter. Fiducial markers made of radio-opaque materials can be detected under fluoroscopy or CT.

FIG. 7 shows that the tip of the guide catheter 701 is placed at the target bladder wall 702. Via the guide catheter 701, a trocar 703 is introduced and advanced through the target bladder wall 702. A folded shunt device 704 is also cannulated through the guide catheter and pushed forward by a pusher catheter 705 to slide up to the target bladder wall. When the trocar contains a longitudinal hollow track along the center of the trocar to allow the aspiration of fluid, the entrance of the trocar and device set into the bladder 706 or peritoneal cavity 707 may be confirmed from the aspirated fluid. Moreover, a flexible guide wire can be introduced through or laterally along the hollow track of the trocar. The guide wire serves as a cannulation track for the shunt device to follow and move back and forth between the body cavities prior to settling at its final implant site.

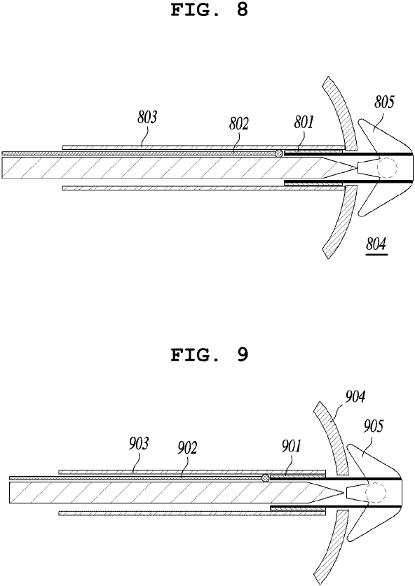

FIG. 8 shows that a self-expandable double-wheel shunt device 801 is pushed forward by a pusher catheter 802 to slide through the guide catheter 803 into the peritoneal space 804. The peritoneal wheel 805 that is uncovered and released out of the guide catheter expands readily by itself because of its intrinsic elastic property. When a guide wire is used to track and access the peritoneal space, the shunt device is pushed forward into the peritoneal space following the guide wire. With a confirmation of the shunt device implanted across the bladder wall, the guide wire is removed.

FIG. 9 illustrates a slight withdrawal of the assembly of the double-wheel shunt device 901, pusher catheter 902, and guide catheter 903 to pull the tip of the guide catheter away from the bladder wall 904. As a consequence of pulling, the self-expanded peritoneal wheel 905 of the double-wheel shunt device splays and apposes to the bladder wall.

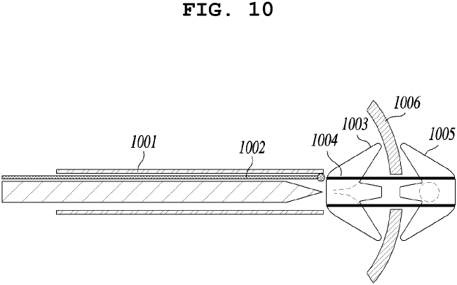

FIG. 10 illustrates a withdrawal of the guide catheter 1001 while keeping the pusher catheter 1002 stationary to uncover the bladder wheel 1003 of the double-wheel shunt device 1004. The self-expanded bladder wheel 1003 and peritoneal wheel 1005 secure and appose the shunt device 1004 to the bladder wall 1006.

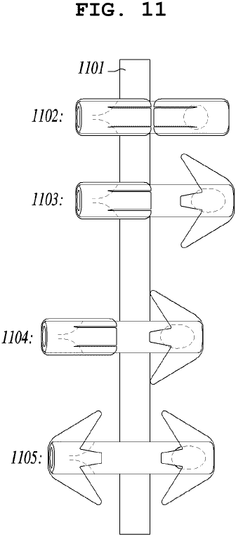

FIG. 11 shows a sequence of implantation in that the wheels of a double-wheel shunt device expand step-by-step to appose to a single-layer wall 1101. Both wheels of the double-wheel shunt device are initially in a folded configuration 1102. One of the double wheels is in an expanded configuration 1103. The expanded wheel is pulled toward the wall in 1104 to appose to the wall and make a room for the folded wing to expand. Both wheels are fully expanded in 1105 across the single-layer wall 1101.

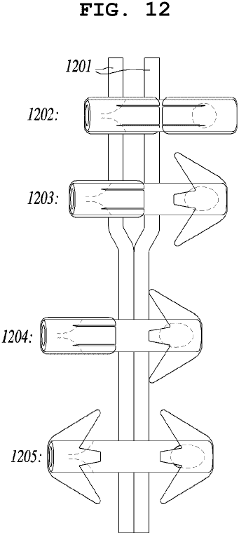

FIG. 12 shows a sequence of implantation in that the wheels of a double-wheel shunt device expand step-by-step and appose to a double-layer wall 1201. Both wheels of the double-wheel shunt device are initially in a folded configuration 1202. One of the double wheels is in an expanded configuration 1203. The expanded wheel is pulled toward the wall in 1204 to bring together the two layers of the wall 1201 to appose to each other and make room for the folded wing to expand. Both wheels are fully expanded in 1205 across the double-layer wall 1201.

FIG. 13 shows that an inflatable double-balloon shunt device 1301 is pushed by a pusher catheter 1302 out of a guide catheter 1303 such that the peritoneal balloon 1304 advances to the peritoneal cavity 1305, while the bladder balloon 1306 remains within the bladder 1307. One-way drainage valve 1308 is located in the central lumen of the shunt device to allow unidirectional flow of ascites from the peritoneal cavity to the bladder and to prevent reflux of urine into the peritoneal cavity. Also advanced with the shunt device is a balloon inflation feeding tube 1309 that is inserted through a one-way balloon valve 1310 into the balloon inflation lumen 1311.

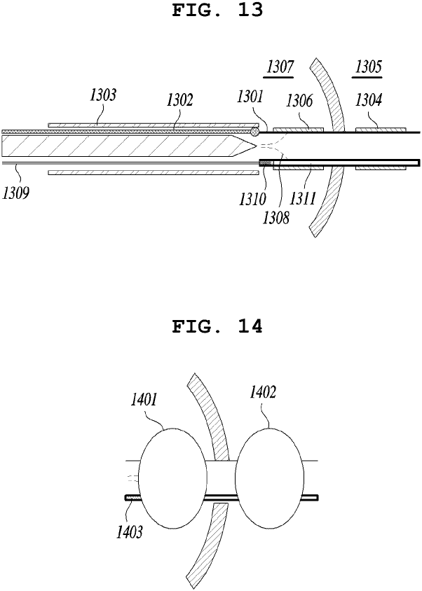

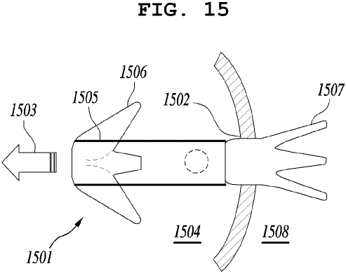

FIG. 14 shows that two balloons 1401 and 1402 of a double-balloon shunt device are inflated by infusion of fluid or air via the balloon inflation feeding tube shown in FIG. 13. After the confirmation of the inflation of the two balloons, the balloon inflation feeding tube, pusher catheter, and guide catheter shown in FIG. 13 are withdrawn and removed. The inflated balloons stabilize and secure the shunt device to resist against the pulling force used to remove the balloon inflation feeding tube. Following the removal of the balloon inflation feeding tube, the balloon inflation lumen is closed by a one-way balloon valve 1403 to keep the balloons inflated.

More than one shunt device can be placed at different locations of the bladder wall to enhance the efficiency of draining ascites, when clinically indicated. When it is desired to remove an implanted double-wheel shunt device, the shunt device in the bladder is directly pulled and removed by using a grasping forceps via cystoscopy. As an illustration, FIG. 15 shows a process of pulling away a double-wheel shunt device 1501 from the shunt implant site 1502 to a direction indicated by the arrow 1503. The shunt device is accessed in the second body cavity 1504 and hauled by grabbing and dragging the hollow drainage tube 1505 or the expanded wheel 1506 of the shunt device. When dragged across the shunt implant site 1502, the expanded wheel 1507 in the first body cavity 1508 is forced to flip and reverse its orientation, slipping through the shunt implant site together with the hollow drainage tube into the second body cavity. The unconstrained shunt device is removed from the second body cavity. For an implanted double-balloon shunt device, a biopsy or snipping tool via cystoscopy is introduced to snip and deflate the bladder balloon that retains the shunt device at the bladder wall. With the deflation of the balloons, the shunt device is freed and removed by using a grasping forceps of a cystoscope.

Although the description of the present invention illustrates mainly the transurethral approach for the implantation of a shunt device, alternative approaches such as the transvesicular and transabdominal insertion approaches are available to access and implant the shunt device depending on clinical indications. For the transvesicular approach, a subcutaneous incision is made at a suitable site on the anterior lower abdomen over the expected suprapubic bladder. A needle is inserted through the abdominal and bladder wall into the bladder lumen. A flexible guide wire is introduced through the bore of the needle which is then removed while the guide wire remains in place. A dilator and introducer sheath are placed over the guide wire and the small needle hole is dilated to a sufficient caliber to accommodate a guide catheter shown in FIG. 6. A shunt device is implanted following the same procedures illustrated in FIGS. 7-14. Following successful implantation of the shunt device, the incision site is closed. For the transabdominal approach, laparoscopy may be used to puncture through the abdominal wall to advance the guide catheter into the peritoneal cavity to the peritoneum abutting the bladder wall. A suitable site at the peritoneum is punctured to create a peritoneo-vesicular fistula. A shunt device with a one-way drainage valve whose flow direction is in reverse to the one-way drainage valve of the transurethral shunt device is implanted following the same procedures illustrated in FIGS. 7-14. Following successful implantation of the shunt device, the incision site is closed.

Furthermore, although the description of the present invention focuses mainly on the peritoneo-vesicular shunt device, the shunt device is applicable to transporting various type of body fluid between two body cavities.

For example, for fetal urinary fluid secondary to lower urinary tract obstruction, the first body cavity is the fetal bladder while the second body cavity is the maternal amniotic sack. For pleural effusion, the first body cavity is the pleural space while the second body cavity is the peritoneal space. For pericardiac effusion, the first body cavity is the pericardiac space while the second body cavity is the pleural space. For a pancreatic cystic fluid collection, the first body cavity is a pancreatic cyst while the second body cavity is the stomach. For a visceral fluid collection, the first body cavity is a visceral fluid collection pocket while the second body cavity is the gastric or intestinal or bladder lumen. For the purpose of draining a body fluid collection to outside of the body, the first body cavity is a body fluid collection pocket while the second body cavity is an external drainage bag outside of the body. In some clinical needs including a continued washout of a body cavity or enteric nutritional support rather than drainage, the aforementioned first body and second cavities may be switched with reversed fluid flow direction.

While the invention has been described with reference to a preferred embodiment, it is to be understood that the foregoing description is merely illustrative, and variations in form, construction, and arrangement may be carried out in other ways without departing from the true spirit and scope of the invention and the following claims.

1. A shunt device transporting fluid between two body cavities comprising:
a lumen-apposing retention system comprising double retention means which is expandable wherein one of the retention means expands to appose to a lumen of a first body cavity and the other retention means expands to appose to a lumen of a second body cavity;
a hollow drainage tube, extending between the first body cavity and the second body cavity, wherein each end of the drainage tube is retained by each of retention means; and
a drainage valve, communicating with a lumen of the drainage tube, wherein the drainage valve is a one-way valve permitting fluid flow from the first body cavity to the second body cavity and preventing fluid flow from the second body cavity to the first body cavity;
wherein a bulk size of the expanded retention means at either side of the body cavities is larger than the cross-sectional coverage of the hollow drainage tube over a shunt aperture area across the two body cavities such that the expanded retention means prevents the hollow drainage tube from slipping out of the shunt aperture into either side of the body cavities.

2. The shunt device of claim 1,
wherein the retention means contains fiducial markers including radio-opaque fiducial markers for tracking the location and position of the retention means.

3. The shunt device of claim 1,
wherein the retention means is double self-expandable wheels.

4. The shunt device of claim 3,
wherein the self-expandable wheels are composed of biocompatible polymer or silicon.

5. The shunt device of claim 1,
wherein the retention means is double inflatable balloons, and the shunt device further comprises:
a small-caliber balloon lumen extending and communicating with the balloons that are inflatable by infusing air or fluid using a small-caliber balloon tube; and
a balloon valve, communicating with the small-caliber balloon lumen, wherein the balloon valve is a one-way valve allowing air or fluid flow to inflate the balloons and preventing a deflation of the balloons once inflated.

6. The shunt device of claim 1,
wherein the first body cavity is a peritoneal cavity and the second body cavity is a bladder.

7. The shunt device of claim 6,
wherein the hollow drainage tube comprises a peritoneal portion located in the peritoneal cavity and a bladder portion located in the bladder.

8. The shunt device of claim 7,
wherein the peritoneal portion of the hollow drainage tube comprises one or more holes to enhance the drainage of ascites.

9. A combined assembly for implanting a self-expandable double-wheel shunt device of claim 3 comprising:
a guide catheter which is steerable to place a tip of the catheter to a target wall(s) of the body cavities that is(are) a target for the implantation of a shunt device;
a trocar which is inserted and advanced through a lumen of the guide catheter to puncture the target wall(s) of the body cavities;
the shunt device which is introduced through the lumen of the guide catheter to the target wall(s) of the body cavities; and
a pusher catheter which pushes the shunt device forward to slide through the lumen of the guide catheter.

10. A combined assembly for implanting an inflatable double-balloon shunt device of claim 5 comprising:
a guide catheter which is steerable to place a tip of the catheter to a target wall(s) of the body cavities that is(are) a target for the implantation of a shunt device;
a trocar which is inserted and advanced through a lumen of the guide catheter to puncture the target wall(s) of the body cavities;
the shunt device which is introduced through the lumen of the guide catheter to the target wall(s) of the body cavities;
a pusher catheter which pushes the shunt device forward to slide through the lumen of the guide catheter; and
a balloon inflation feeding tube which extends from outside of a patient to an entrance of the small-caliber balloon lumen, wherein the balloon inflation feeding tube fits into the small-caliber balloon lumen to inflate balloons of the shunt device by infusing fluid or air using a syringe.

11. The combined assembly of claim 9,
wherein the combined assembly further comprises a guide wire which is introduced through or laterally along the hollow lumen of the trocar to serve as a cannulation track for a shunt device to move across the body cavities.

12. The combined assembly of claim 10,
wherein the combined assembly further comprises a guide wire which is introduced through or laterally along the hollow lumen of the trocar to serve as a cannulation track for a shunt device to move across the body cavities.

13. The combined assembly of claim 11,
wherein the guide catheter contains fiducial markers including radio-opaque fiducial markers to track the location and position of the guide catheter.

14. The combined assembly of claim 12,
wherein the guide catheter contains fiducial markers including radio-opaque fiducial markers to track the location and position of the guide catheter.

15. A method to implant a self-expandable double-wheel peritoneo-vesicular shunt device from the combined assembly of claim 9 via urethra comprising:
placing the guide catheter in a bladder and steering the tip of the guide catheter to a bladder wall that is a target for the implantation of the shunt device and that juxtaposes a peritoneum in direct contact with ascites within a peritoneal space;
introducing and advancing the trocar through the guide catheter to the target bladder wall and puncturing the target bladder wall;
introducing and pushing the shunt device forward by the pusher catheter through the lumen of the guide catheter to advance and expose only a peritoneal portion of the shunt device out of the guide catheter into a peritoneal cavity, which allows the folded peritoneal wheel of the shunt device to unbind and self-expand;
pulling back slightly the assembly of the guide catheter, pusher catheter, and shunt device to place the tip of the guide catheter within the bladder while maintaining the expanded peritoneal wheel to appose to the bladder wall;
withdrawing the guide catheter slightly while keeping the pusher catheter stationary to uncover and unbind a bladder portion of the shunt device out of the guide catheter in the bladder, which allows the folded bladder wheel of the shunt device to unbind and self-expand so that the shunt device is stabilized and secured across the bladder wall; and
removing the pusher catheter, trocar, and guide catheter.

16. A method to implant an inflatable double-balloon peritoneo-vesicular shunt device from the combined assembly of claim 10 via urethra comprising:
placing the guide catheter in a bladder and steering the tip of the guide catheter to a bladder wall that is a target for the implantation of the shunt device and that juxtaposes a peritoneum in direct contact with ascites within a peritoneal space;
introducing and advancing the trocar through the guide catheter to the target bladder wall and puncturing the target bladder wall;
introducing and pushing the shunt device forward by the pusher catheter through the lumen of the guide catheter to advance and expose only a peritoneal portion of the shunt device out of the guide catheter into a peritoneal cavity, which allows a peritoneal balloon of the double-balloon shunt to unbind;
withdrawing the guide catheter slightly while keeping the pusher catheter stationary to uncover and unbind a bladder balloon of the shunt device out of the guide catheter in the bladder;
inflating the bladder balloon and peritoneal balloon of the shunt device by infusing fluid or air via the balloon inflation feeding tube to stabilize and secure the shunt device across the bladder wall;
removing the balloon inflation feeding tube from the balloon valve that closes the small-caliber balloon lumen; and
removing the pusher catheter, trocar, and guide catheter.

17. The method of claim 15,
which further comprises:
introducing and advancing a guide wire through or laterally along the hollow lumen of the trocar to serve as a cannulation track for the shunt device to access the peritoneal space, after the puncturing the target bladder wall, and
removing the guide wire, after the shunt device being secured across the bladder wall.

18. The method of claim 16,
which further comprises:
introducing and advancing a guide wire through or laterally along the hollow lumen of the trocar to serve as a cannulation track for the shunt device to access the peritoneal space, after the puncturing the target bladder wall, and
removing the guide wire, after the shunt device being secured across the bladder wall.

19. A method to remove an implanted double-wheel shunt device of claim 3 comprising:
accessing and grasping a side of the shunt device located in the second body cavity by using a grasping forceps;
pulling the hollow drainage tube of the shunt device to slip through the shunt implant site into the second body cavity and forcing the expanded wheel of the shunt device in the first body cavity to flip and reverse its orientation and slip through the shunt implant site into the second body cavity; and
further pulling and removing the freed shunt device from the second body cavity.

20. A method to remove an implanted double-balloon shunt device of claim 5 comprising:
accessing and snipping the balloon of the shunt device located in the second body cavity by using a biopsy or snipping tool to deflate the balloons in both body cavities;
grasping and pulling the deflated and freed shunt device by using a grasping forceps to slip through the shunt implant site from the first body cavity to the second body cavity; and
further pulling and removing the released shunt device from the second body cavity.
